(19) United States
(12) Patent Application Publication
(10) Pub. No.: US 2010/0256626 A1
(43) Pub. Date: Oct. 7, 2010

(54) EYE THERAPY SYSTEM

(75) Inventors: David Muller, Boston, MA (US); Thomas Ryan, Waltham, MA (US); Ronald Scharf, Waltham, MA (US)

Correspondence Address: NIXON PEABODY, LLP, 300 S. Riverside Plaza, 16th Floor, CHICAGO, IL, 60606-6613, US

(73) Assignee: Avedro, Inc., Waltham, MA (US)

(21) Appl. No.: 12/753,465

(22) Filed: Apr. 2, 2010

Related U.S. Application Data
(60) Provisional application No. 61/166,009, filed on Apr. 2, 2009.

(51) Int. Cl. A61B 18/18; A61N 1/00

(52) U.S. Cl. 606/34; 607/53

(57) ABSTRACT

Embodiments according to aspects of the present invention provide a single convenient and versatile tool that allows an operator to apply energy to the cornea according to different patterns to suit different treatment cases, without requiring multiple applicators or interchangeable components. An electrical energy applicator in one embodiment extends from a proximal end to a distal end. The energy conducting applicator includes, at the proximal end, a connection to one or more electrical energy sources. The energy conducting applicator directs electrical energy from the one or more electrical energy sources to the distal end. The distal end is positionable at a surface of an eye. The energy conducting applicator includes at least three selectable conductors coupled to the one or more electrical energy sources. The selectable conductors define an outer conductor and an inner conductor being separated by a gap. Each of the selectable conductors are independently activated or deactivated according to a pattern of electrical energy to be applied to the eye.

### CROSS-REFERENCE TO RELATED APPLICATIONS

This application claims the benefit of priority from U.S. Provisional Application No. 61/166,009, filed Apr. 2, 2009, which is hereby incorporated by reference in its entirety.

### BACKGROUND OF THE INVENTION

1. Field of the Invention

The invention pertains to the field of keratoplasty and, more particularly, to a system and method for applying thermokeratoplasty.

2. Description of Related Art

A variety of eye disorders, such as myopia, keratoconus, and hyperopia, involve abnormal shaping of the cornea. Keratoplasty reshapes the cornea to correct such disorders. For example, with myopia, the shape of the cornea causes the refractive power of an eye to be too great and images to be focused in front of the retina. Flattening aspects of the cornea's shape through keratoplasty decreases the refractive power of an eye with myopia and causes the image to be properly focused at the retina.

Invasive surgical procedures, such as laser-assisted in-situ keratomileusis (LASIK), may be employed to reshape the cornea. However, such surgical procedures typically require a healing period after surgery. Furthermore, such surgical procedures may involve complications, such as dry eye syndrome caused by the severing of corneal nerves.

Thermokeratoplasty, on the other hand, is a noninvasive procedure that may be used to correct the vision of persons who have disorders associated with abnormal shaping of the cornea, such as myopia, keratoconus, and hyperopia. Thermokeratoplasty may be performed by applying electrical energy in, for example, the microwave band or radio frequency (RF) band. In particular, microwave thermokeratoplasty may employ a near field microwave applicator to apply energy to the cornea and raise the corneal temperature. At about 60° C., the collagen fibers in the cornea shrink. The onset of shrinkage is rapid, and stresses resulting from this shrinkage reshape the corneal surface. Thus, application of heat energy in circular or ring-shaped patterns may cause aspects of the cornea to flatten and improve vision in the eye.

### SUMMARY

Embodiments according to aspects of the present invention provide a single convenient and versatile tool that allows an operator to apply energy to the cornea according to different patterns to suit different treatment cases, without requiring multiple applicators or interchangeable components.

An electrical energy applicator in one embodiment extends from a proximal end to a distal end. The energy conducting applicator includes, at the proximal end, a connection to one or more electrical energy sources. The energy conducting applicator directs electrical energy from the one or more electrical energy sources to the distal end. The distal end is positionable at a surface of an eye. The energy conducting applicator includes at least three selectable conductors coupled to the one or more electrical energy sources. The selectable conductors define an outer conductor and an inner conductor being separated by a gap. Each of the selectable conductors are independently activated or deactivated according to a pattern of electrical energy to be applied to the eye.

In operation, the distal end of the electrical energy applicator is positioned at a surface of an eye, and the selectable conductors are independently activated or deactivated to define an outer conductor and an inner conductor separated by a gap. Electrical energy is applied through the electrical energy applicator to the eye according to the pattern.

An electrical energy applicator in another embodiment extends from a proximal end to a distal end. The energy conducting applicator includes, at the proximal end, a connection to one or more electrical energy sources. The energy conducting applicator directs electrical energy from the one or more electrical energy sources to the distal end. The distal end is positionable at a surface of an eye. The energy conducting applicator includes an outer conductor and an inner conductor extending to the distal end. The inner conductor is disposed within the outer conductor and separated from the outer conductor by a gap. The outer conductor includes one or more outer segments. The inner conductor includes a plurality of inner segments. Each of the one or more outer segments and the plurality of inner segments are activated or deactivated according to a pattern of electrical energy to be applied to the eye.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates a system for applying heat to a cornea of an eye to cause reshaping of the cornea.

FIG. 2A illustrates a high resolution image of a cornea after heat has been applied.

FIG. 2B illustrates another high resolution image of the cornea of FIG. 2A.

FIG. 2C illustrates a histology image of the cornea of FIG. 2A.

FIG. 2D illustrates another histology image of the cornea of FIG. 2A.

FIG. 3A illustrates a system with an applicator that includes differently dimensioned conductors for applying thermokeratoplasty according to aspects of the present invention.

FIG. 3B illustrates another view of the system of FIG. 3A.

FIG. 4A illustrates a system with an applicator that includes segmented conductors for applying thermokeratoplasty according to further aspects of the present invention.

FIG. 4B illustrates another view of the system of FIG. 4A.

FIG. 5A illustrates a system with an applicator that includes segmented conductors for applying thermokeratoplasty according to still further aspects of the present invention.

FIG. 5B illustrates another view of the system of FIG. 5A.

FIG. 6A illustrates a system with an applicator that includes segmented conductors for applying thermokeratoplasty according to still further aspects of the present invention.

FIG. 6B illustrates another view of the system of FIG. 6A.

### DESCRIPTION

Embodiments according to aspects of the present invention provide an applicator that includes a series of differently dimensioned conductors for applying thermokeratoplasty. In one embodiment, the applicator includes a series of concentric, differently dimensioned conductors that allow energy to be applied to a cornea in varying patterns. In particular, the applicator provides a single convenient and versatile tool that allows an operator to apply energy to the cornea according to different patterns to suit different treatment cases, without requiring multiple applicators or interchangeable components. Moreover, the applicator may be particularly advantageous when multiple applications of energy according to different patterns are required to achieve the desired change in the shape of a cornea.

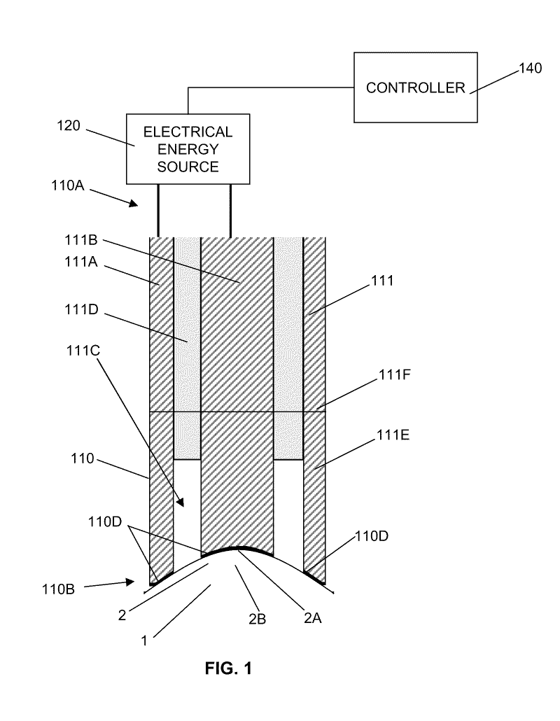

FIG. 1 illustrates an example system for applying energy to a cornea 2 of an eye 1 to generate heat and cause reshaping of the cornea. In particular, FIG. 1 shows an applicator 110 with an electrical energy conducting element 111 that is operably connected to an electrical energy source 120, for example, via conventional conducting cables. The electrical energy conducting element 111 extends from a proximal end 110A to a distal end 110B of the applicator 110. The electrical energy conducting element 111 conducts electrical energy from the source 120 to the distal end 110B to apply heat energy to the cornea 2, which is positioned at the distal end 110B. In particular, the electrical energy source 120 may include a microwave oscillator for generating microwave energy. For example, the oscillator may operate at a microwave frequency range of about 400 MHz to about 3000 MHz, and more specifically at a frequency of about 915 MHz or about 2450 MHz, which has been safely used in other applications. As used herein, the term “microwave” corresponds to a frequency range from about 10 MHz to about 10 GHz.

As further illustrated in FIG. 1, the electrical energy conducting element 111 may include two microwave conductors 111A and 111B, which extend from the proximal end 110A to the distal end 110B of the applicator 110. In particular, the conductor 111A may be a substantially cylindrical outer conductor, while the conductor 111B may be a substantially cylindrical inner conductor that extends through an inner passage extending through the conductor 111A. With the inner passage, the conductor 111A has a substantially tubular shape. The inner and the outer conductors 111A and 111B may be formed, for example, of aluminum, stainless steel, brass, copper, other metals, coated metals, metal-coated plastic, metal alloys, combinations thereof, or any other suitable conductive material.

With the concentric arrangement of conductors 111A and 111B, a substantially annular gap 111C of a selected thickness is defined between the conductors 111A and 111B. The annular gap 111C extends from the proximal end 110A to the distal end 110B. A dielectric material 111D may be used in portions of the annular gap 111C to separate the conductors 111A and 111B. The distance of the annular gap 111C between conductors 111A and 111B determines, in part, the penetration depth of microwave energy into the cornea 2 according to established microwave field theory. Thus, the microwave conducting element 111 receives, at the proximal end 110A, the electrical energy generated by the electrical energy source 120, and directs microwave energy to the distal end 110B, where the cornea 2 is positioned.

In general, the outer diameter of the inner conductor 111B may be selected to achieve an appropriate change in corneal shape, i.e. keratometry, induced by the exposure to electrical energy. Meanwhile, the inner diameter of the outer conductor 111A may be selected to achieve a desired gap between the conductors 111A and 111B. For example, the outer diameter of the inner conductor 111B ranges from about 2 mm to about 10 mm while the inner diameter of the outer conductor 111A ranges from about 2.1 mm to about 12 mm. In some systems, the annular gap 111C may be sufficiently small, e.g., in a range of about 0.1 mm to about 2.0 mm, to minimize exposure of the endothelial layer of the cornea (posterior surface) to elevated temperatures during the application of heat by the applicator 110. The pattern in which the energy is applied to the cornea 2 depends on the dimensions of the outer conductor 111A and the inner conductor 111B. For example, the energy may be applied according to a ring of a selected diameter, where the diameter is determined by the dimensions of the inner conductor 111A and the outer conductor 111B.

A controller 140 may be employed to selectively apply the energy any number of times according to any predetermined or calculated sequence. In addition, the energy may be applied for any length of time. Furthermore, the magnitude of energy being applied to the eye feature (e.g., the cornea 2) may also be varied. Adjusting such parameters for the application of energy determines the extent of changes that are brought about within the cornea 2. Of course, the system attempts to limit the changes in the cornea 2 to an appropriate amount of shrinkage of collagen fibrils in a selected region. When employing microwave energy to generate heat in the cornea 2, for example with the applicator 110, the microwave energy may be applied with low power (e.g., of the order of 40 W) and in long pulse lengths (e.g., of the order of one second). However, other systems may apply the microwave energy in short pulses. In particular, it may be advantageous to apply the microwave energy with durations that are shorter than the thermal diffusion time in the cornea. For example, the microwave energy may be applied in pulses having a higher power in the range of about 500 W to about 3 kW and a pulse duration in the range of about 5 milliseconds to about one second.

Referring again to FIG. 1, at least a portion of each of the conductors 111A and 111B may be coated or covered with an electrical insulator to minimize the concentration of electrical current in the area of contact between the corneal surface (epithelium) 2A and the conductors 111A and 111B. In some systems, the conductors 111A and 111B, or at least a portion thereof, may be coated or covered with a material that can function both as an electrical insulator and/or a thermal conductor.

In the system illustrated in FIG. 1, a dielectric layer 110D is disposed along the distal end 111B of the applicator 110 to protect the cornea 2 from electrical conduction current that would otherwise flow into the cornea 2 via conductors 111A and 111B. Such current flow may cause unwanted temperature effects in the cornea 2 and interfere with achieving a maximum temperature within the collagen fibrils in a mid-depth region 2B of the cornea 2. Accordingly, the dielectric layer 110D is positioned between the conductors 111A and 111B and the cornea 2. The dielectric layer 110D may be sufficiently thin to minimize interference with microwave emissions and thick enough to prevent superficial deposition of electrical energy by flow of conduction current. For example, the dielectric layer 110D may be a biocompatible material deposited to a thickness of about 10-100 micrometers, preferably about 50 micrometers. As another example, the dielectric layer 110D can be a flexible sheath-like structure of biocompatible material that covers the conductors 111A and 111B at the distal end 110B and extends over a portion of the exterior wall of the outer conductor 111B. As still a further example, the dielectric layer 110D can include a first flexible sheath-like structure of biocompatible material that covers the distal end of the inner conductor 111A and a second flexible sheath-like structure of biocompatible material that covers the distal end of the outer conductor 111B. As yet another example, the dielectric layer 110D can be applied as a coating of dielectric material on the conductors.

In general, an interposing layer, such as the dielectric layer 110D, may be employed between the conductors 111A and 111B and the cornea 2 as long as the interposing layer does not substantially interfere with the strength and penetration of the microwave radiation field in the cornea 2 and does not prevent sufficient penetration of the microwave field and generation of a desired heating pattern in the cornea 2. The dielectric material may be elastic (e.g., polyurethane, silastic, combinations thereof and/or the like) or nonelastic (e.g., Teflon®, ceramics of various dielectric constants, polyimides, combinations thereof and/or the like). The dielectric material may have a fixed dielectric constant or varying dielectric constant by mixing materials or doping the sheet, the variable dielectric being spatially distributed so that it may affect the microwave hearing pattern in a customized way. The thermal conductivity of the material may have fixed thermal properties (e.g., thermal conductivity or specific heat), or may also vary spatially, through mixing of materials or doping, and thus provide a means to alter the heating pattern in a prescribed manner. Another approach for spatially changing the heating pattern is to make the dielectric sheet material of variable thickness. The thicker region will heat less than the thinner region and provides a further means of spatial distribution of microwave heating.

During operation, the distal end 110B of the applicator 110 as shown in FIG. 1 is positioned on or near the corneal surface 2A. Preferably, the applicator 110 makes direct contact with the corneal surface 2A. In particular, such direct contact positions the conductors 111A and 111B at the corneal surface 2A (or substantially near the corneal surface 2A if there is a thin interposing layer between the conductors 111A and 111B and the corneal surface 2A). Accordingly, direct contact helps ensure that the pattern of microwave heating in the corneal tissue has substantially the same shape and dimension as the gap 111C between the two microwave conductors 111A and 111B.

The system of FIG. 1 is provided for illustrative purposes only, and other systems may be employed to apply energy to generate heat and reshape the cornea. Other systems are described, for example, in U.S. patent application Ser. No. 12/208,963, filed Sep. 11, 2008, which is a continuation-in-part application of U.S. patent application Ser. No. 11/898,189, filed on Sep. 10, 2007, the contents of these applications being entirely incorporated herein by reference. According to U.S. patent application Ser. No. 12/208,963, a cooling system may be employed in combination with the applicator 110 to apply coolant to the cornea 2 and determine how the energy is applied to the cornea 2.

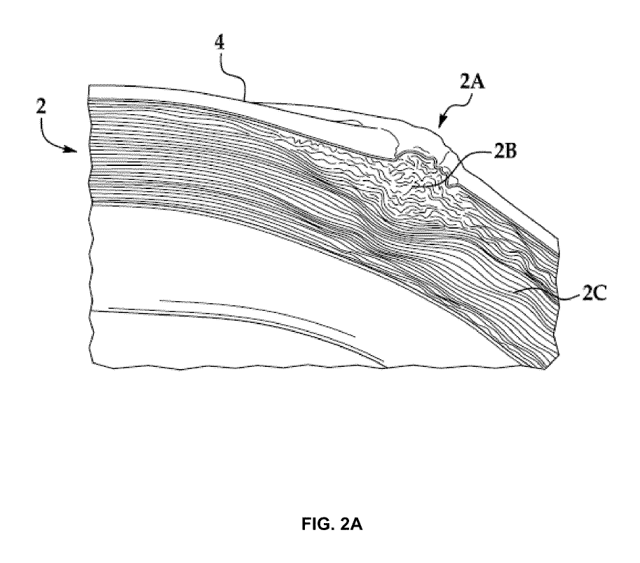

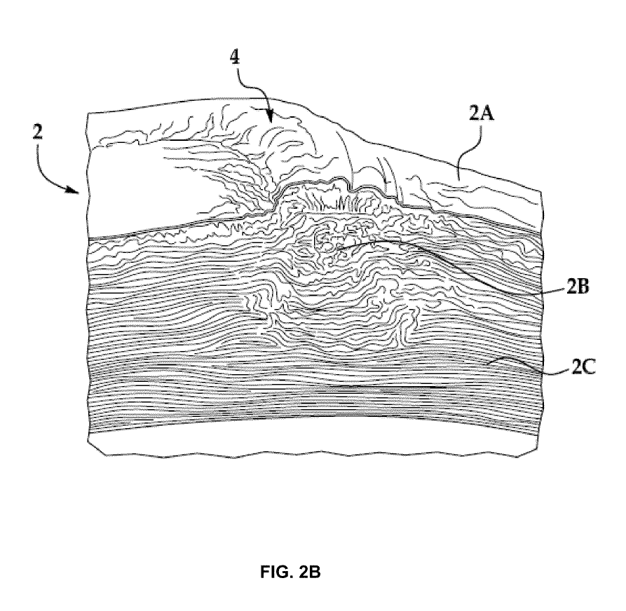

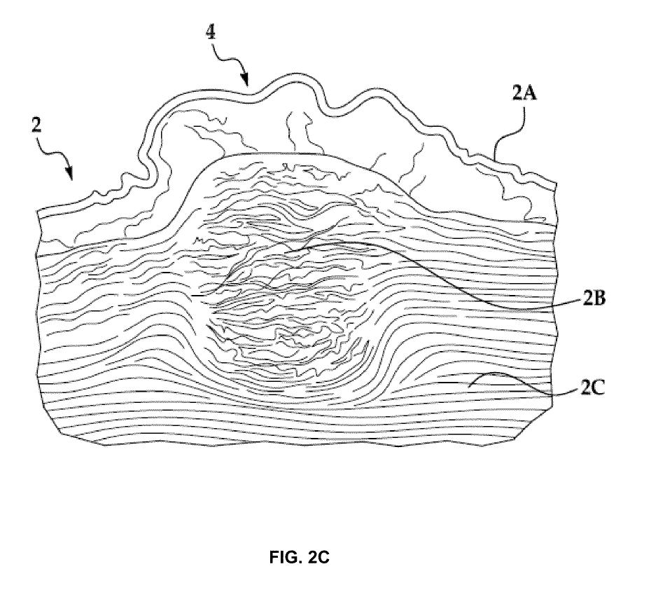

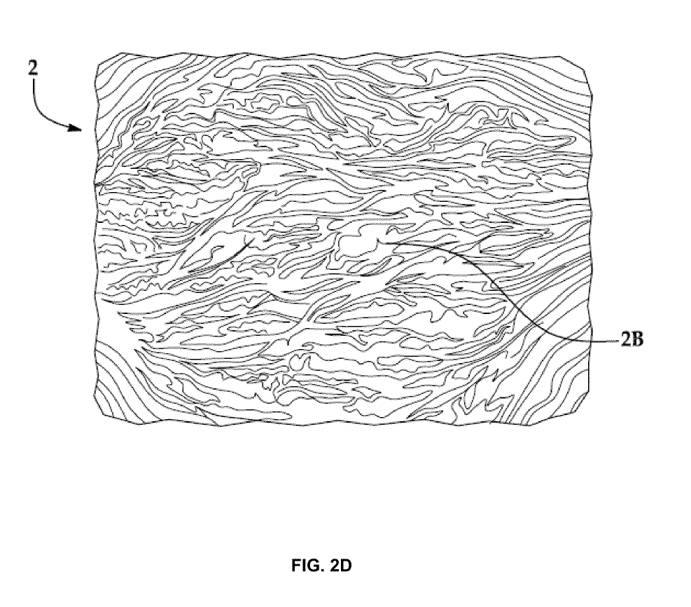

FIGS. 2A-D illustrate an example of the effect of applying heat to corneal tissue with a system for applying heat, such as the system illustrated in FIG. 1. In particular, FIGS. 2A and 2B illustrate high resolution images of cornea 2 after heat has been applied. As FIGS. 2A and 2B show, a lesion 4 extends from the corneal surface 2A to a mid-depth region 2B in the corneal stroma 2C. The lesion 4 is the result of changes in corneal structure induced by the application of heat as described above. These changes in structure result in an overall reshaping of the cornea 2. It is noted that the application of heat, however, has not resulted in any heat-related damage to the corneal tissue.

As further illustrated in FIGS. 2A and 2B, the changes in corneal structure are localized and limited to an area and a depth specifically determined by an applicator as described above. FIGS. 2C and 2D illustrate histology images in which the tissue shown in FIGS. 2A and 2B has been stained to highlight the structural changes induced by the heat. In particular, the difference between the structure of collagen fibrils in the mid-depth region 2B where heat has penetrated and the structure of collagen fibrils outside the region 2B is clearly visible. Thus, the collagen fibrils outside the region 2B remain generally unaffected by the application of heat, while the collagen fibrils inside the region 2B have been rearranged and formed new bonds to create completely different structures. In other words, unlike processes, like orthokeratology, which compress areas of the cornea to reshape the cornea via mechanical deformation, the collagen fibrils in the region 2B are in an entirely new state.

As described previously with reference to FIG. 1, the pattern in which the energy is applied to the cornea 2 and the resulting change in corneal shape depend on the dimensions of the outer conductor 111A and the inner conductor 111B. For example, the application of energy in a ring-shaped pattern depends on the inner diameter of the outer conductor 111A and the outer diameter of the inner conductor 111B. Thus, applicators having different dimensions must be available to allow an operator to produce desired shape changes on a case-by-case basis. One possible approach would make several separate applicators available, where each applicator is configured with different fixed dimensions. Alternatively, as described in U.S. patent application Ser. No. 12/208,963, the applicator 110 as shown in FIG. 1 may include interchangeable components. In particular, the applicator 110 may include a replaceable end piece 111E that defines the energy conducting element 111 at the distal end 110B. The end piece 111E is removably attached at a connection 111F with the rest of the energy conducting element 111 using any conductive coupling that permits energy to be sufficiently conducted to the cornea 2. For example, the end piece 111E may be received via threaded engagement, snap connection, other mechanical interlocking, or the like. Accordingly, end pieces 111E having different dimensions and/or shapes may be employed with a single applicator 110. As such, a single applicator 110 may deliver energy to the cornea 2 according to varying patterns defined by replaceable end pieces 111E with different dimensions. Other aspects of end pieces employable with the applicator 110 are described, for example, in U.S. patent application Ser. No. 12/018,473, filed Jan. 23, 2008, the contents of which are incorporated herein by reference.

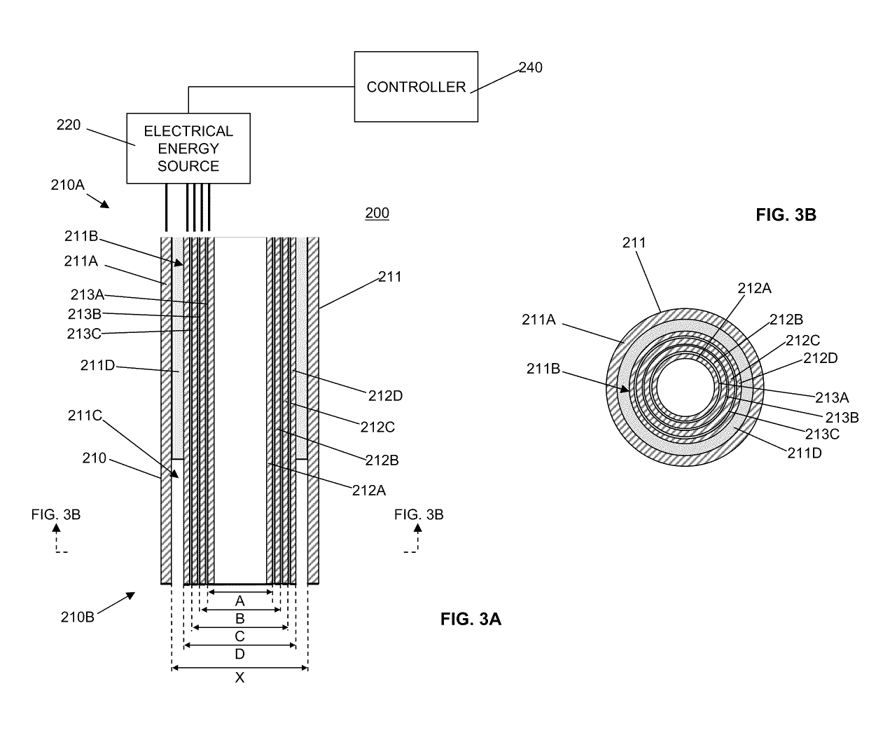

Rather than employing changeable end pieces 111E to apply energy according to different patterns, embodiments, as illustrated in FIGS. 3A-B, may employ an energy conducting element 211 that includes a series of differently dimensioned inner conductors for applying energy to a cornea of an eye to cause reshaping of the cornea 2. Similar to the system 100 of FIG. 1, the system 200 shown in FIG. 3A includes an applicator 210 with an electrical energy conducting element 211 that is operably connected to an electrical energy source 220. The electrical energy conducting element 211 extends from a proximal end 210A to a distal end 210B of the applicator 210. The electrical energy conducting element 211 conducts electrical energy (e.g., microwave energy) from the energy source 220 to the distal end 210B to apply heat energy to the cornea, which is positioned at the distal end 210B. A controller 240 may be employed to control operation of the applicator 210 in a manner similar to the controller 140 described previously with reference to FIG. 1.

As further illustrated in FIG. 3A, the electrical energy conducting element 211 operates via two conductors 211A and 211B, which extend from the proximal end 210A to the distal end 210B. The conductors 211A and 211B may be formed, for example, of aluminum, stainless steel, brass, copper, other metals, coated metals, metal-coated plastic, metal alloys, combinations thereof, or any other suitable conductive material. The conductor 211A may be a substantially tubular outer conductor similar to the outer conductor 111A shown in FIG. 1, while the conductor 211B is an inner conductor that extends through an inner passage extending through the conductor 211A. Unlike the inner conductor 111B shown in FIG. 1, however, the inner conductor 211B includes a series of separate conductors 212A-D that allow the outer conductor 211A to be used in combination with inner conductors of differing dimensions.

As shown in FIG. 3A-B, substantially cylindrical conductors 212A-D are arranged in a concentric configuration. The conductors 212A-D may also be concentric with respect to the outer conductor 211A as well as to each other. As such, the inner conductor 211B includes several conductors 212A-D with different outer diameters A, B, C, and D, respectively, where each conductor 212A, 212B, 212C, and 212D provides differently dimensioned ring-shaped patterns when combined with the inner diameter of the outer conductor 211A. Although the example described herein may include four conductors 212A-D, it is understood that other embodiments may include any number of conductors in a similar series configuration.

In particular, the conductor 212A extends through a passageway in the conductor 212B. Although FIGS. 3A-B may show that the conductor 212A is substantially tubular, it is understood that the conductor 212A does not have to be tubular and/or may include other structures or features within the passageway. To prevent or inhibit conduction of electrical current between the conductors 212A and 212B, the conductors 212A and 212B are separated by a substantially annular gap, and a layer 213A, formed from a dielectric such as those described previously, is disposed between the conductors 212A and 212B. The combination of the conductors 212A and 212B then extends through a passageway in the conductor 212C. A dielectric layer 213B is also disposed in a substantially annular gap separating the conductors 212B and 212C. Similarly, the combination of the conductors 212A, 212B, and 212C extends through a passageway in the conductor 212D, and a dielectric layer 213C is disposed in a substantially annular gap separating the conductors 212C and 212D. Meanwhile, the combination of the conductors 212A-D (i.e., the inner conductor 211B) extends through the outer conductor 211A. In addition, a dielectric material 211D may be disposed in portions of the annular gap between the outer conductor 211A and the conductor 212D. In some embodiments, the dielectric layers 213A-C may be formed as a part of sheath-like structures positioned over the outer surface of the conductors 212A-C, respectively.

In addition to the substantially annular gaps defined between the conductors 212A and 212B, 212B and 212C, and 212C and 212D, a substantially annular gap 211C of varying dimension is defined between the outer conductor 211A and each conductor 212A, 212B, 212C, or 212D. The annular gap 211C extends to the distal end 210B. As shown in FIG. 3A, the inner diameter of the outer conductor 211A is X. Thus, the gap 211C between the outer conductor 211A and the conductor 212A has an annular thickness of (X-A). The gap 211C between the outer conductor 211A and the conductor 212B has an annular thickness of (X-B). The gap 211C between the outer conductor 211A and the conductor 212C has an annular thickness of (X-C). The gap 211C between the outer conductor 211A and the conductor 212D has an annular thickness of (X-D). The outer diameters A, B, C, and D may range, in increasing dimension, from about 2 mm to about 10 mm, while the inner diameter of the outer conductor 211A may range from about 2.1 mm to about 12 mm. As described previously, the gap 211C determines the penetration depth of energy into the cornea, so the gap 211C may be sufficiently small to minimize exposure of the endothelial layer of the cornea (posterior surface) to elevated temperatures during the application of heat by the applicator 210.

As FIG. 3A illustrates further, the outer conductor 211A and each of the conductors 212A-D can be coupled to the electrical energy source 220. In operation, electrical energy from the energy source 220 may be conducted from the proximal end 210A to the distal end 210B via the outer conductor 211A and one of the conductors 212A-D. Thus, the selected conductor 212A, 212B, 212C, or 212D conducts the electrical energy for the inner conductor 211B in a manner similar to the inner conductor 111B discussed previously. In some embodiments, the controller 240 may be employed to select and activate the conductor 212A, 212B, 212C, or 212D. In other embodiments, the conductor 212A, 212B, 212C, or 212D may be selected or activated, for example, by manually coupling the selected conductor to the source 220 while leaving the other conductors decoupled from the source 220.

Thus, the single applicator 210 provides four different outer conductor and inner conductor pairings, where each pairing provides an annular gap 211C of different dimensions. By coupling the outer conductor 211A with the appropriate conductor 212A, 212B, 212C, or 212D, one of the outer diameters A, B, C, or D for the inner conductor 211B may be selected to achieve the desired annular gap 211C and an appropriate change in corneal shape. In particular, the selected outer diameter A, B, C, or D determines the diameter of the ring-shaped pattern by which energy is applied to the cornea.

Accordingly, the applicator 210 provides a single convenient and versatile tool that allows an operator to apply energy to the cornea according to different patterns to suit different treatment cases, without requiring multiple applicators or interchangeable components. Although the applicator 210 may be employed for a single application of energy according to a single outer conductor/inner conductor pair, the applicator 210 may be particularly advantageous when multiple applications of energy according to multiple patterns are required to achieve the desired change in the shape of the cornea. For example, energy may be incrementally applied to the cornea in precise and measured steps in multiple ring-shaped patterns. An example of a multi-step approach is described in U.S. Patent Ser. No. 61/098,489, filed on Sep. 19, 2008, the contents of which are entirely incorporated herein by reference. In general, energy may be applied multiple times according to different patterns and pulses, i.e., duration and magnitude, to achieve the desired shape change. Indeed, in some embodiments, an asymmetric or non-annular shape change, for example to treat astigmatism, may be effected by multiple applications of energy in different ring-shaped patterns that are centered at different areas of the cornea.

Additionally or alternatively, the outer conductor 211A may include a series of separate conductors that allow the inner conductor 211B to be used in combination with outer conductors of differing dimensions. Indeed, one embodiment may provide a series of evenly spaced concentric conductors, any of which may be selectively activated to act as a pair of conducting elements.

In yet other embodiments, a combination of two or more inner conductors may be energized simultaneously with the single outer conductor to further influence the heating pattern. In additional embodiments, the series of conductors may also be slightly recessed relative to the outer conductor such that the shape of the eye is matched. For example, one to four conductors may be in contact with the eye at varying recessed positions to either conform to the eye shape or to create a predetermined cornea shape during treatment. In further embodiments, some of the conductors may remain un-energized but may be moved into contact with the cornea according to a predefined shape, while a neighboring conductor is energized. This technique allows the cornea surface, including portions which are not treated, to be effectively pre-shaped.

As explained above, in some systems, the conductors 211A and 211B, or at least a portion thereof, may be coated with or covered by a material that can function both as an electrical insulator as well as a thermal conductor. The material may be a dielectric layer employed along the distal end 210B of the applicator 210 to protect the cornea 2 from electrical conduction current that would otherwise flow into the cornea 2 via conductors 211A and 211B. As an example, the dielectric layer can be a flexible sheath-like structure of biocompatible material that covers the conductors 211A and 211B at the distal end 210B and extends over a portion of the exterior wall of the outer conductor 211B. As another example, the dielectric layer can include a first flexible sheath-like structure of biocompatible material that covers the distal end of the inner conductor 211A and a second flexible sheath-like structure of biocompatible material that covers the distal end of the outer conductor 211B. As still a further example, the dielectric layer can be formed as a plurality of sheath-like structures that are individually positioned over the outer surface of the outer conductor 211A and each of the inner conductors 212A-D. As yet another example, the dielectric layer can be a coating of dielectric material applied to the conductors.

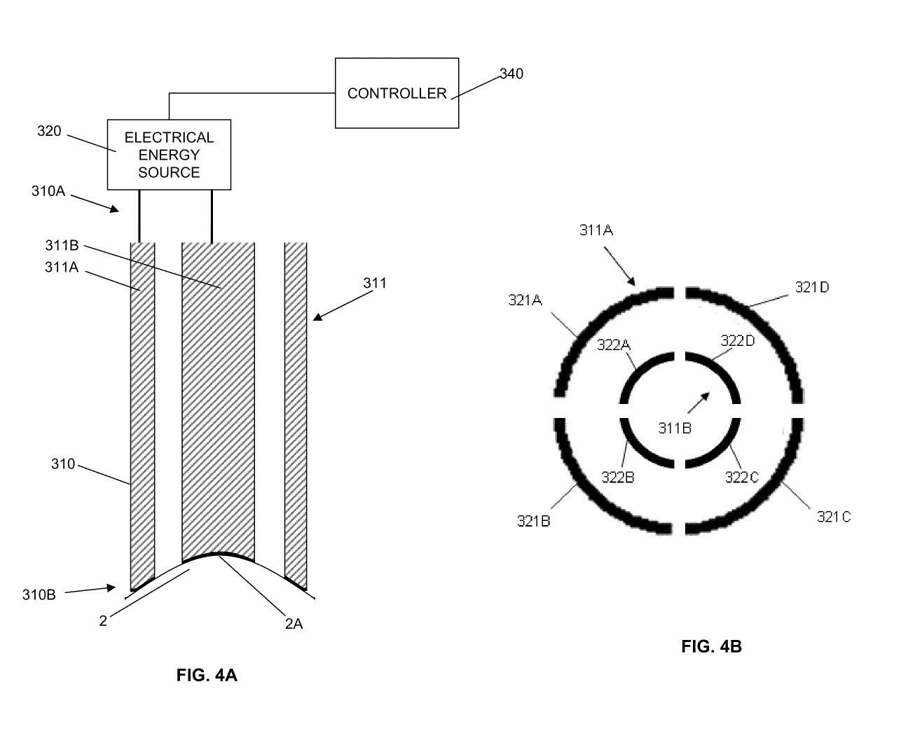

FIGS. 4A-B illustrates a system 300 with an applicator 310 according to further aspects of the present invention. Similar to the systems 100 and 200 described above, the system 300 shown in FIG. 4A includes an applicator 310 with an electrical energy conducting element 311 that is operably connected to an electrical energy source 320. The electrical energy conducting element 311 extends from a proximal end 310A to a distal end 310B of the applicator 310. The electrical energy conducting element 311 conducts electrical energy (e.g., microwave energy) from the energy source 320 to the distal end 310B to apply heat energy to the cornea 2, which is positioned at or near the distal end 310B. A controller 340 may be employed to control operation of the applicator 310 in a manner similar to the controller 140 and 240 described previously.

Like the energy conducting element 111 and 211, the energy conducting element 311 includes an outer conductor 311A and an inner conductor 311B that extend along a longitudinal axis from a proximal end 310A to a distal end 310B. However, the outer conductor 311A is defined at the distal end 310B by a plurality of outer conductor segments 321A-D, and the inner conductor 311B is defined at the distal end 310B by a plurality of inner conductor segments 322A-D. In other words, the outer conductor 311A and the inner conductor 311B are each configured to contact the corneal surface 2A with more than one component.

The conductor segments 321A-D and 322A-D may be formed, for example, of aluminum, stainless steel, brass, copper, other metals, coated metals, metal-coated plastic, metal alloys, combinations thereof, or any other suitable conductive material. To prevent or inhibit conduction of electrical current between adjacent outer conductor segments 321A-D, the segments 321A-D are separated by a gap, and a layer formed from a dielectric such as those described previously, is disposed between the segments 321A-D. Similarly, to prevent or inhibit conduction of electrical current between adjacent inner conductor segments 322A-D, the segments 322A-D are separated by a gap, and a layer formed from a dielectric such as those described previously, is disposed between the segments 322A-D. In addition, a dielectric material may be disposed in portions of the annular gap between the outer conductor 311A and the inner conductor 311B.

As explained above, in some systems, the conductors 311A and 311B, or at least a portion thereof, may be coated with or covered by a material that can function both as an electrical insulator as well as a thermal conductor. The material may be a dielectric layer employed along the distal end 310B of the applicator 310 to protect the cornea 2 from electrical conduction current that would otherwise flow into the cornea 2 via conductors 311A and 311B. As an example, the dielectric layer can be a flexible sheath-like structure of biocompatible material that covers the conductors 311A and 311B at the distal end 310B and extends over a portion of the exterior wall of the outer conductor 311B. As another example, the dielectric layer can include a first flexible sheath-like structure of biocompatible material that covers the distal end of the inner conductor 311A and a second flexible sheath-like structure of biocompatible material that covers the distal end of the outer conductor 311B. As still a further example, the dielectric layer can be formed as a plurality of sheath-like structures that are individually positioned over the outer surface of each of the conductor segments 321A-D and 322A-D of the conductors 312A and 312B, respectively. As yet another example, the dielectric layer can be a coating of dielectric material applied to the conductors.

Each of the outer conductor segments 321A-D and each of the inner conductor segments 322A-D are coupled to the energy source such that at least a portion (and preferably all) of the conductor segments 321A-D and 322A-D can be independently activated and/or deactivated. In operation, electrical energy from the energy source is conducted from the proximal end 310A to the distal end 310B of the conducting element 311 via one or more of the outer conductor segments 321A-D and one or more of the inner conductor segments 322A-D. Thus, the selected conductor segments 321A-D and 322A-D conduct the electrical energy for the conductors 311A and 311B, respectively, in a manner similar to the conductors 111A-B and 211A-B discussed previously.

In some embodiments, a controller may be employed to select and activate one or more of the conductor segments 321A, 321B, 321C, 321D, 322A, 322B, 322C, and/or 322D. In other embodiments, the conductor 321A, 321B, 321C, 321D, 322A, 322B, 322C, and/or 322D may be selected or activated, for example, by manually coupling the selected conductor segment(s) to the energy source 320 while leaving the other conductor segment(s) decoupled from the energy source 320. When some conductor segments 321A-D and 322A-D are activated (i.e., supplied with energy from the energy source 320) and other conductor segments 321A-D and 322A-D are not activated, part of the circumference (e.g.,)90-180° of the outer conductor 311A and/or the inner conductor 311B no longer applies heat energy to the cornea surface 2. Thus, the pattern of heating is biased away from the non-activated region(s).

Accordingly, a single applicator including the conducting element 311 provides numerous different conductor segment 321A-D and 322A-D combinations, where each combination applies a different pattern of energy to a cornea. In particular, the selected combination of conductor segments 321A-D and 322A-D can provide asymmetric or non-annular energy patterns, which may be advantageous in treating specific eye conditions or disorders, such as astigmatism.

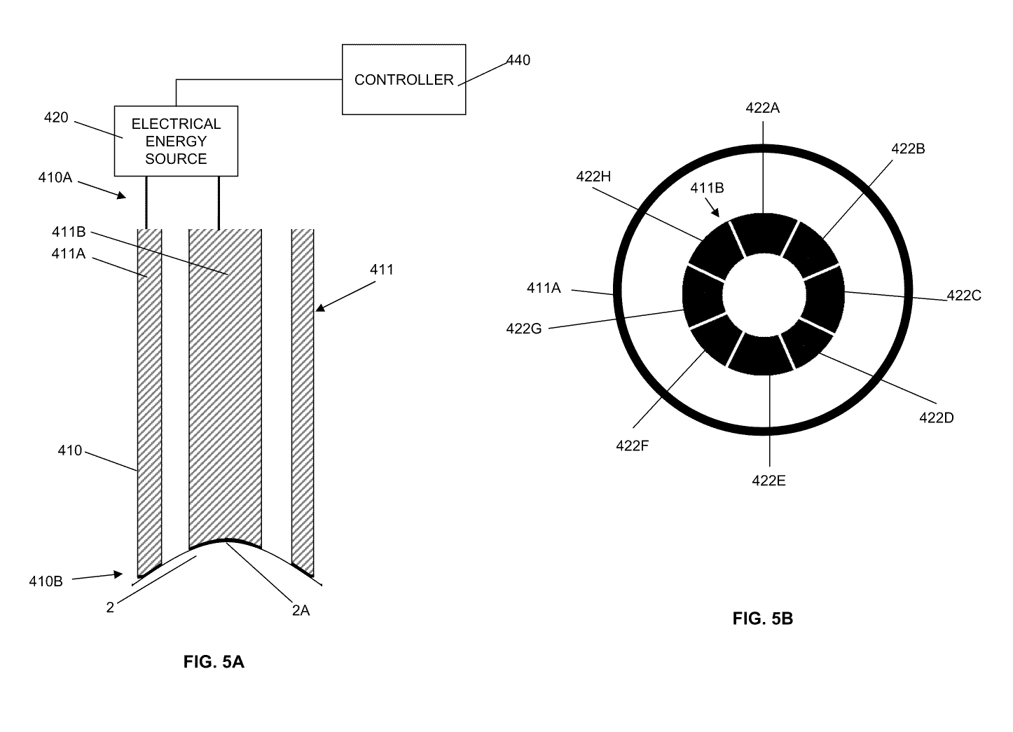

FIGS. 5A-B illustrate another embodiment according to the aspects of the present invention. System 400 is substantially the same as system 300 described above with reference to FIGS. 4A-B, except system 400 includes an electrical conducting element 411 having a cylindrical outer conductor 411A and an inner conductor 411B defined at the distal end 410B by eight inner conductor segments 422A-H. Accordingly, some inner conductor segments 422A-H can be activated, while other inner conductor segments 422A-H are not activated as described above with reference to FIGS. 4A-B. The resulting energy patterns produced by system 400 are particularly useful for the treatment of astigmatism.

The magnitude and angle of astigmatism can be viewed as a superposition of two astigmatic components defined by the following equations:

C₊=C/2 cos(2A) (1)

Cₓ=C/2 sin(2A) (2)

Seq=S+C/2 (3)

where C₊is the astigmatic component in the 0/90 degree orientation, Cₓ is the astigmatic component in the 45/135 degree orientation, Seq is the spherical equivalent, C is an astigmatism in Diopters, A is the angle of astigmatism in degrees, and S is the spherical component (of refractive error).

To treat astigmatism, the spherical equivalent, the C₊component and the Cₓ component are calculated. The spherical equivalent can be treated by activating all inner conductor segments 422A-H to apply energy to the cornea 2. The C₊component and the Cₓ component can then be treated by selectively activating and deactivating particular inner conductor segments 422A-H to apply an asymmetric or non-annular pattern of energy to the cornea 2.

For example, if the C₊component is a positive number, the C₊component can be treated by activating inner conductor segments 422B, 422C, 422D, 422F, 422G, and 422H, while not activating (or deactivating) inner conductor segments 422A and 422E. If the C₊component is a negative number, the C₊component can be treated by activating inner conductor segments 422A, 422B, 422D, 422E, 422F, and 422H, while not activating (or deactivating) inner conductor segments 422C and 422G. If the Cₓ component is a positive number, the Cₓ component can be treated by activating inner conductor segments 422A, 422C, 422D, 422E, 422G, and 422H, while not activating (or deactivating) inner conductor segments 422B and 422F. And if the Cₓ component is a negative number, the Cₓ component can be treated by activating inner conductor segments 422A, 422B, 422C, 422E, 422F, and 422G, while not activating (or deactivating) inner conductor segments 422D and 422H.

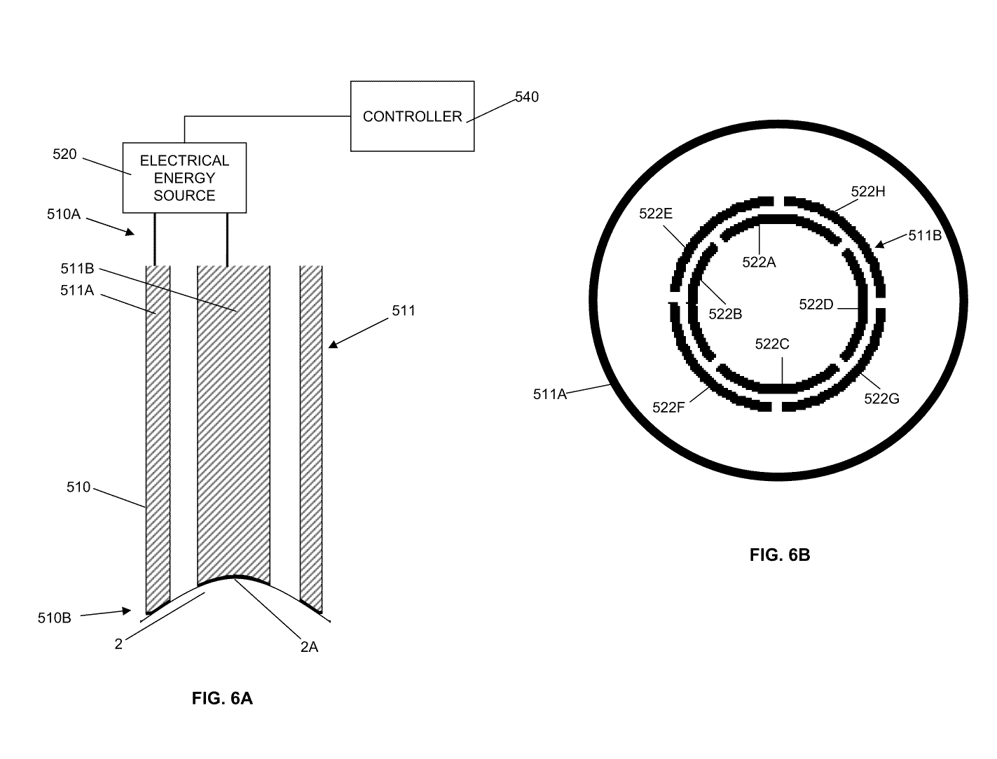

FIGS. 6A-B illustrate still another embodiment according to the aspects of the present invention. System 500 is substantially the same as system 400 described above with reference to FIGS. 5A-B, including an electrical conducting element 511 having a cylindrical outer conductor 511A and an inner conductor 511B defined at the distal end 510B by eight inner conductor segments 522A-H, except the eight inner conductor segments 522A-H are configured in two concentric rings. Accordingly, some inner conductor segments 522A-H can be activated, while other inner conductor segments 522A-H are not activated as described above with reference to FIGS. 4A-B and 5A-B. The resulting energy patterns produced by the system 500 can be used to treat astigmatism like the system 400 by activating one set of electrodes to treat the 0/90 degree astigmatic component and activating another set of electrodes to treat the 45/135 degree astigmatic component.

Accordingly, the applicators described herein provide a single convenient and versatile tool that allows an operator to apply energy to the cornea according to different patterns to suit different treatment cases, without requiring multiple applicators or interchangeable components. Although the applicators described herein may be employed for a single application of energy according to a single outer conductor/inner conductor pair, the applicators may be particularly advantageous when multiple applications of energy according to multiple patterns are required to achieve the desired change in the shape of the cornea. In general, energy may be applied multiple times according to different patterns and pulses, i.e., duration and magnitude, to achieve the desired shape change.

Although the embodiments described herein may employ concentric conductors, other embodiments may employ any combination of concentric and non-concentric conductors to produce different shapes and dimensions for the gaps between conductors. Similarly, although the embodiments described herein can apply energy to the cornea according to an annular pattern defined by an applicator (such as the applicator 210), the pattern in other embodiments is not limited to a particular shape. For example, the inner conductor may include a series of conductors with an elliptical profile to apply energy according to elliptical patterns of varying dimensions. Indeed, energy may be applied to the cornea in non-annular patterns. Examples of the non-annular patterns by which energy may be applied to the cornea are described in U.S. patent Ser. No. 12/113,672, filed on May 1, 2008, the contents of which is entirely incorporated herein by reference. Additionally, as shown for the applicators 310, 410 and 510, non-annular patterns can be applied by selectively activating and/or deactivating particular conductors or segments of conductors.

Although the embodiments described herein may employ conductor segments that are shaped as sections of a cylinder, the conductor segments can have different shapes and sizes. For example, the conductor segments can have a cylindrical, pin-like shape, or any other polygonal shape. It is contemplated that in some embodiments, the segments may include a combination of different shapes and sizes. Additionally, while the embodiments described herein may employ conductors including four or eight conductor segments, the conductors can include any number of segments. While the embodiment of FIG. 4B illustrates the segments of the inner conductor aligned with the segments of the outer conductor, in some embodiments, the segments may not be aligned.

Although embodiments above may refer to one energy source and to one controller, it is understood that more than one respective energy source and/or more than one controller may be employed to operate an applicator according to aspects of the present invention. For example, referring to the embodiment of FIG. 3, each of the conductors 211A, 212A, 212B, 212C, or 212D may be coupled to a dedicated energy source. The conductors 211A, 212A, 212B, 212C, or 212D and their respective energy sources may be selectively activated by one controller. Alternatively, each of the conductors 211A, 212A, 212B, 212C, or 212D may each be selectively activated by a dedicated controller. In general, any number of conductors or conductor segments may be coupled to any number of energy sources and any number of controllers to deliver an appropriate amount energy for an appropriate duration according to a desired pattern.

Furthermore, the controller(s) described above may be a programmable processing device that executes software, or stored instructions, and that may be operably connected to the other devices described above. In general, physical processors and/or machines employed by embodiments of the present invention for any processing or evaluation may include one or more networked or non-networked general purpose computer systems, microprocessors, field programmable gate arrays (FPGAs), digital signal processors (DSPs), micro-controllers, and the like, programmed according to the teachings of the exemplary embodiments of the present invention, as is appreciated by those skilled in the computer and software arts. The physical processors and/or machines may be externally networked with the image capture device, or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits (ASICs) or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present invention may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present inventions for performing all or a portion (if processing is distributed) of the processing performed in implementing the inventions. Computer code devices of the exemplary embodiments of the present inventions can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present inventions can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

And while the above embodiments are described as applying energy to the cornea, it is understood that in some embodiments the energy may be applied to other features of an eye.

While the present invention has been described in connection with a number of exemplary embodiments, and implementations, the present inventions are not so limited, but rather cover various modifications, and equivalent arrangements.

We claim:

1. A system for applying therapy to an eye, the system comprising:
one or more electrical energy sources; and
an electrical energy conducting element extending from a proximal end to a distal end, the energy conducting element operably connected to the one or more electrical energy sources at the proximal end and adapted to direct electrical energy to the distal end, the distal end being positionable at a surface of an eye, the energy conducting element including at least three selectable conductors, the selectable conductors being coupled to the one or more electrical energy sources, each of the plurality of selectable conductors being independently activated or deactivated, the plurality of selectable conductors defining an outer conductor and an inner conductor being separated by a gap, the selectable conductors being activated or deactivated according to a pattern of electrical energy to be applied to the eye.

2. The system of claim 1, wherein an outermost one of the selectable conductors is activated to define the outer conductor and at least one of the remaining selectable conductors is activated to define the inner conductor, the gap being defined by a distance between the outermost selectable conductor and the at least one remaining selectable conductor that is activated.

3. The system of claim 1, wherein the outer conductor is defined by more than one of the selectable conductors.

4. The system of claim 1, wherein the gap is substantially annular.

5. The system of claim 1, wherein the selectable conductors are substantially cylindrical.

6. The system of claim 4, wherein the plurality of selectable conductors are concentric.

7. The system of claim 1, wherein each selectable conductor is separated from adjacent ones of the plurality of selectable conductors by a space and a dielectric material is disposed in the space between adjacent ones of the plurality of selectable conductors.

8. The system of claim 1 further comprising a controller operable to activate at least one of the plurality of selectable conductors by controlling the supply of energy from the one or more electrical energy sources to each of the plurality of selectable conductors.

9. The system of claim 1, wherein the pattern is asymmetric or non-annular.

10. A method for applying therapy to an eye, the method comprising:
positioning an electrical energy conducting element at a surface of an eye, the energy conducting element being operably connected to one or more electrical energy sources at a proximal end and extending to a distal end, the energy conducting element including at least three selectable conductors, the selectable conductors being coupled to the one or more electrical energy sources;
independently activating or deactivating each of the plurality of selectable conductors to define an outer conductor and an inner conductor separated by a gap, the outer conductor and the inner conductor providing a pattern of electrical energy to be applied to the eye; and
applying electrical energy through the electrical energy conducting element to the eye according to the pattern.

11. The method of claim 10, wherein activating or deactivating each of the plurality of selectable conductors comprises:
activating an outermost one of the selectable conductors to define the outer conductor; and
activating at least one of the remaining selectable conductors to define the inner conductor,
wherein the gap is defined by a distance between the outermost selectable conductor and the at least one remaining selectable conductor that is activated.

12. The system of claim 10, wherein activating or deactivating each of the plurality of selectable conductors comprises activating or deactivating each of a plurality of outermost ones of the selectable conductors to define the outer conductor.

13. The method of claim 10, wherein the gap is substantially annular.

14. The method of claim 10, wherein the selectable conductors are substantially cylindrical.

15. The method of claim 14, wherein the plurality of selectable conductors are concentric.

16. The method of claim 14, wherein each selectable conductor is separated from adjacent ones of the plurality of selectable conductors by a space and a dielectric material is disposed in the space between adjacent ones of the plurality of selectable conductors.

17. The method of claim 10, wherein the pattern is asymmetric or non-annular.

18. A system for applying therapy to an eye, the system comprising:
one or more electrical energy source; and
an electrical energy conducting element extending from a proximal end to a distal end, the energy conducting element operably connected to the one or more electrical energy source at the proximal end and adapted to direct electrical energy to the distal end, the energy conducting element including:
an outer conductor extending to the distal end, the outer conductor including one or more outer segment; and
an inner conductor extending to the distal end and disposed within the outer conductor, the inner conductor including a plurality of inner segments, the outer conductor and the inner conductor being separated by a gap,
wherein each of the one or more outer segment and the plurality of inner segments are activated or deactivated according to a pattern of electrical energy to be applied to the eye.

19. The system of claim 18, wherein each of the one or more outer segment and each of the plurality of inner segments are shaped as sections of a cylinder.

20. The system of claim 18, wherein each of the one or more outer segment and each of the plurality of inner segments have a polygonal shape at the distal end.

21. The system of claim 18, wherein the plurality of inner segments are configured as concentric rings.

22. The system of claim 18 further comprising one or more controllers operable to activate at least one of the outer segments and at least one of the inner segments by controlling the supply of energy from the one or more electrical energy sources to each of the outer segments and each of the inner segments.

23. The system of claim 18, wherein the pattern is asymmetric or non-annular.

24. The system of claim 18, wherein each of the inner segments is separated from adjacent ones of the inner segments by a space and a dielectric material is disposed in the space between adjacent ones of the inner segments.

25. A method for applying therapy to an eye, the method comprising:
positioning an electrical energy conducting element at a surface of an eye, the energy conducting element being operably connected to one or more electrical energy sources at a proximal end and extending to a distal end, the energy conducting element including:
an outer conductor extending to the distal end; and
an inner conductor extending to the distal end and disposed within the outer conductor, the inner conductor including a plurality of inner segments, the plurality of inner segments being coupled to the one or more electrical energy source such that each of the plurality of inner segments can be independently activated and deactivated, the outer conductor and the inner conductor being separated by a gap;
independently activating or deactivating each of the plurality of inner segments to define a pattern of electrical energy to be applied to the eye;
applying electrical energy through the electrical energy conducting element to the eye according to the pattern.

26. The method of claim 25, wherein the outer conductor includes a plurality of outer segments coupled to the one or more electrical energy source such that each of the plurality of outer segments can be independently activated and deactivated.

27. The method of claim 25, wherein the pattern is nonannular or asymmetric.
